(19) United States
(12) Patent Application Publication
(10) Pub. No.: US 2012/0029285 A1
(43) Pub. Date: Feb. 2, 2012

(54) INSTRUMENTS

(76) Inventor: David Main, Leeds (GB)

(21) Appl. No.: 12/962,275

(22) Filed: Dec. 7, 2010

(30) Foreign Application Priority Data
Jul. 28, 2010 (GB) GB1012609.2

(51) Int. Cl. A61B 1/00

(52) U.S. Cl. 600/129; 600/139

(57) ABSTRACT

An endoscopic instrument having a first part moveable from a first configuration to a second, different configuration, the instrument including a stiffener extending across the region that the first part is moveable from the first to the second configuration, the stiffener being moveable from a first position in which movement between the first and second configurations is inhibited and a second position in which movement between the first and second configurations is permitted. A method of using an endoscopic instrument.

### CROSS-REFERENCE TO RELATED APPLICATION

This Application claims priority to GB 1012609.2 filed Jul. 28, 2010 and entitled “INSTRUMENTS”, the contents of which are incorporated by reference herein.

### TECHNICAL FIELD AND BACKGROUND OF THE INVENTION

The present invention relates to endoscopic surgical instruments and a method of using such instruments. EP 0 623 004 discloses a surgical instrument comprising a retractor. It is an object of the invention to overcome the disadvantages of the prior art.

### BRIEF SUMMARY OF THE INVENTION

According to one aspect of the present invention, an endoscopic instrument including an elongate portion having a first part moveable from a first configuration to a second, different configuration the instrument including a stiffener extending across the region that the first part is moveable from the first to the second configuration, the stiffener being moveable from a first position in which movement between the first and second configurations is inhibited and a second position in which movement between the first and second configurations is permitted.

According to a further aspect of the present invention, an endoscopic instrument including an elongate portion having a first part moveable from a first configuration to a second, different configuration the instrument including a stiffener extending across the region that the first part is moveable from the first to the second configuration, the stiffener being moveable from a first position in which movement between the first and second configurations is inhibited and a second position in which the stiffener is moved in the elongate direction of the instrument such that the stiffener does not extend across the first and second parts and in which movement between the first and second configuration is permitted, the stiffener being slidably mounted within the elongate portion, the instrument including a piercer at the distal end.

According to a further aspect of the present invention a method of using an endoscopic instrument comprising piercing the wall of a body with a piercer at the distal end of the instrument and inserting an elongate extent of the instrument through the wall and moving a stiffener that is slidably mounted within the elongate portion and that extends across a first part that is moveable from a first configuration to a second different configuration from a first position in which the movement of the first part is inhibited to a second position in which the stiffener does not extend across the first part and in which the movement of the first part is permitted.

### DESCRIPTION OF THE FIGURES

FIG. 1 is a side view of a surgical instrument comprising a surgical retractor 1 with an end 2 in a straight configuration;

FIG. 2 is a view of the end 2 of the retractor shown in FIG. 1 in a straight hook configuration;

FIG. 3 is a view of an end 2 of a retractor similar to that shown in FIG. 1 in an angled hook configuration;

FIG. 4 is a schematic perspective view of one of the segments 3 at the end 2 of the retractor shown in FIG. 1;

FIG. 5 is a front view of a retractor;

FIG. 6 is a front view of another embodiment of a retractor;

FIG. 7 is a perspective view of a different embodiment of a retractor;

FIGS. 8a-i are sequential views showing how the configuration of FIG. 7 is formed when the wire 10 is pulled;

FIGS. 9a-i are sequential views showing how the configuration of FIG. 7 is formed when the wire 10 is pulled when using control members;

FIG. 10 is a longitudinal cross-section of the instrument that can form the shape of FIG. 7 and FIG. 11 is a detail of that figure;

FIG. 12 is a longitudinal cross sectional view of part of the instrument showing the wire 10 and the control member and FIG. 13 is cross-section 13-13 of FIG. 12; and

FIG. 14 is a perspective view of part of the instrument.

### DETAILED DESCRIPTION OF THE INVENTION

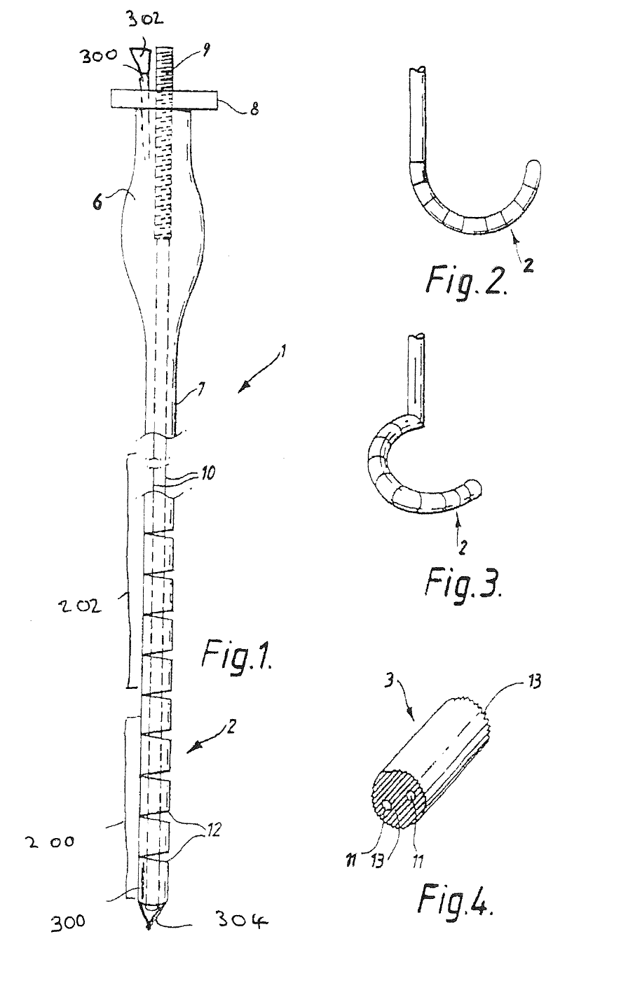

FIG. 1 shows a retractor 1 having a handle 6 which is connected to the end 2 via a hollow rod 7. In use, with the end in the configuration shown in the drawing, a cut is made in the abdominal wall and a sealed port is located in the cut. Then the end 2 and part of the rod 7 are fed through a tube passing through the port in the abdominal wall. The surgeon is then able to manipulate the retractor by the handle 6 and change the configuration of the end 2 into the straight hook shape shown in FIG. 2 by rotating a knurled actuating nut 8.

The nut 8 is threadably connected to a screw member 9 whereby, when the nut 8 is rotated in a clockwise direction, looking from the free end of the handle, the screw member 9 is caused to move translationally away from the end 2. A loop of wire 10 is connected at its free ends to the member 9, and both sides of the loop pass through openings 11 in each segment 3. Accordingly as the wire 10 moves further into the rod 7 the segments are caused to tighten against each other.

As the segments 3 bear against each other they are caused to move out of the axial extent of the rod as the end faces 12 of each segment are formed at a slight angle to the perpendicular to the axis of the rod. In FIG. 1 the upwardly facing surfaces of each segment are parallel with each other as are the downwardly facing surfaces. Accordingly adjacent faces come into abutment with each other as the wire is tightened, and they take up the configuration shown in FIG. 2 in which a straight, substantially rigid hook which subtends approximately 180° is formed. Accordingly in the position shown in FIG. 1, each face extends at an angle of approximately 10° to the axis of the tube.

In order for the segments to take up the shape shown in FIG. 3, in which a substantially rigid hook which subtends approximately 180° in a direction generally at right angles to the axis of the rod, the face of at least one of the segments is angled differently. For instance, when the end 2 is in the relaxed position and extends generally in line with the axis of the rod 7, the uppermost segment faces the rod with a face extending at 45° to the axis of the rod, and the rod may be correspondingly angled at its end. Thus when the wire is tightened, the segment adjacent to the rod is caused to turn through 90°. The remaining upper and lower faces of the other segments may be parallel to each other in the configuration shown in FIG. 1 as previously described.

The hooks shown in FIGS. 2 and 3 can be used to displace or hold the organs in the required position.

To release the segments from the configuration shown in FIG. 2 or 3 the nut 8 is rotated in the opposite direction to release the tension in the wire. The wire is sufficiently strong, and the distance between the segments sufficiently small for the flexure of the wire to hold the segments generally straight for ease of insertion or removal when the hook configuration is not required. As the wire is threaded through two openings in each segment the strength of the wire and the close proximity of the segments prevents any significant relative turning of the segments around the longitudinal extent of the end 2.

The face of each segment which is caused to abut against another part of the retractor when in the hook configuration is formed with styrations 13 which are parallel to each other and parallel to adjacent styrations such that co-operating faces do not tend to slip in a rotational or translational sense.

Each of the embodiments of the retractors shown may be operated as described in relation to FIGS. 1 to 4. Accordingly only the differences will be described. In addition, each retractor is able to have a straight configuration to enable the retractor to be inserted or removed and only the second configurations are shown in which each adjacent segment abuts each other to inhibit further bending.

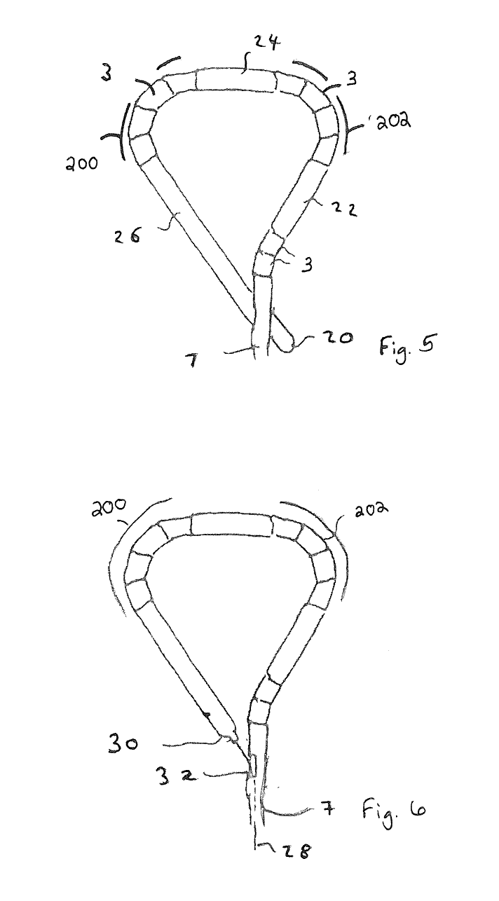

In FIG. 5 there are four segments 3 adjacent to the hollow rod 7 and four at each of the further corners. Long segments 22 and 24 extend between the short segments and a longer segment 26 has its tip 20 extending back under the rod 7. Ideally the tip 20 should be concealed in the view shown by the hollow rod.

This arrangement has advantages over the segmental arrangements shown in FIGS. 2 and 3 in that no twisting of the retractor about the shaft 7 occurs if the retractor is urged in a direction out of the plane shown or into the plane shown. In addition at least part of the tip 20 is concealed by the rod 7 or can trail the rod 7 thus effecting less trauma.

FIG. 6 has the same general shape of that of FIG. 5. However the shaft 7 includes a further control cable 28 that is connected to the tip 30 of the elongate portion. The cable exits the shaft 7 just short of the first series of segments.

In use, either before the segments are tensioned by the wires 10 to take up the configuration shown, or after, or during at least part of that tensioning or any combination thereof the control cable 28 is tensioned to draw the tip 30 towards the shaft 7. A recess 32 may be provided in the shaft 7 in which the tip 30 may be drawn into and held by the cable.

This configuration allows greater force to be applied with less trauma being provided than that of FIG. 5. In addition the retractor can be urged in either direction to equal advantage as the configuration is symmetrical from the front and back.

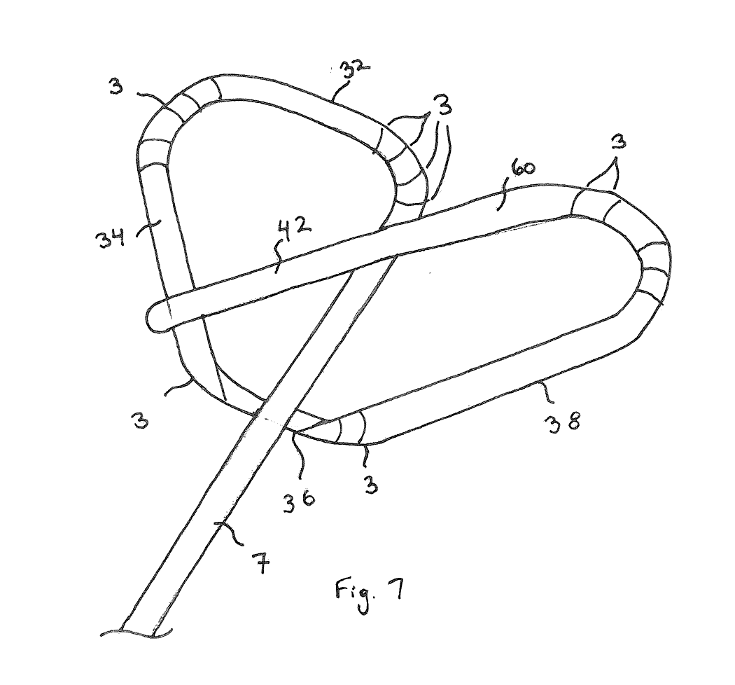

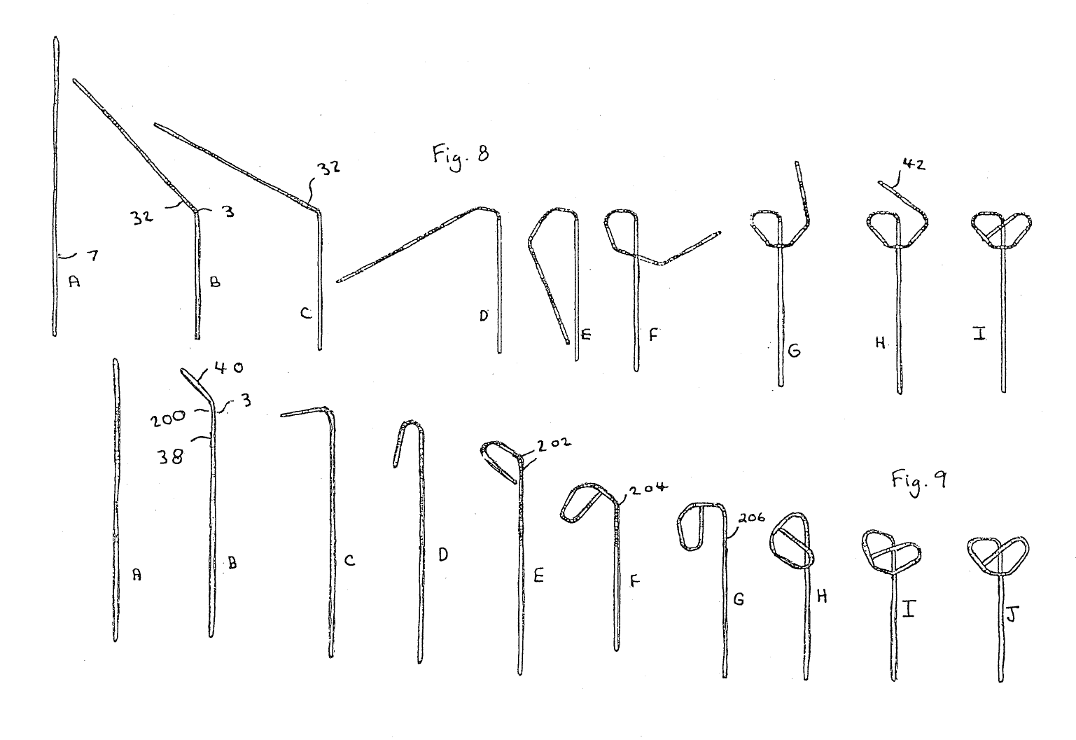

Whilst the angles that the short segment of FIGS. 5 and 6 allow the retractor to turn are approximately 45°. 135° and 135° respectively the embodiment of FIG. 7 is more complicated.

In FIG. 7, starting from the rod 7, four short segments 3 allow the next long segment 32 to extend at 90° to the extent of the rod. Then six short segments 3 cause an even longer segment 34 to extend back towards the rod 7 at an angle of 45° to the rod. Then two short segments cause the “shortest” long segment 36 to cross over the rod 7, possibly in contact therewith, before two further short segments cause a further turn of 45° for the next long segment 38. Then eight short segments 3 cause a further turn of 180°. This brings two long segments 40 and 42 back over the rod 7 with the end of the segment 42 being tucked under the long segment 34.

The long segments 40 and 42 are connected by angled faces that allow the segment 40 to be inclined upwardly as it extends towards the rod with the segment 42 being inclined downwardly as it extends away from the rod 7. The movement of the segments 40 and 42 can be coordinated to take place as the end of the instrument moves back over the shaft towards the segment 34. Alternatively the segments 40 and 42 may be fixed together to form an angled suit such that they can not move relative to each other. The segments 40 and 42 may be urged against the long segment 32 and the short segments 3 adjacent to the rod 7 as the segments 40 and 42 are being moved into place such that relative flexure of those parts occurs. When the joint between the segments 40 and 42 pass the segments 3 adjacent to the rod they spring back to allow the segment 42 to pass beneath the segment 34 and to maintain the shape shown under flexure with those parts crossing the rod being urged against the rod. Furthermore, that binding force may also cause the segment 36 to be biased. The biasing forces may be assisted by the angled slope of the segment 42 sliding along the segment 34 and pushing further against the segment 34 as the segment 42 slides further beneath the segment 34.

The configuration of FIG. 7 affords stability and strength in either direction. Furthermore trauma is reduced because of the considerable cross sectional area provided by the retractor or both sides of the shaft 7.

Although not shown in the drawing of FIG. 7, the end segment 42 may be connected to the shaft 7 by a control cable. The control cable may be tensioned to assist in the retractor leaving the straight configuration. As the wires tension the segments and as the retractor takes up the shape shown the cable may be tensioned or relaxed to assist in the shape being taken up.

As the retractor crosses the rigid rod in FIGS. 5 and 7 (and as the retractor is fixed in FIG. 6) when the rod is urged towards a liver with the cross parts being located between the rod and liver a rigid retractor is provided with a broad area of even force being applied.

FIGS. 8a-i are sequential views showing how the configuration of FIG. 7 is formed when the wire 10 is pulled. The first part of the instrument that bends is the proximal part between the rod 7 and the long segment 32 that changes shape from FIGS. 8a to 8b to 8c. When the short segments 3 abut each other to prevent further rotation at that location the adjacent short segments then cause the shape to change from FIGS. 8c to 8d. It can be seen that further tightening causes turning of the instrument to progress towards the proximal end possibly with abutment of distally located segment prior to adjacent proximal segments commencing to turn or possibly with a partial turn of distal segments prior to adjacent proximal segments commencing to turn.

It can be seen though that there is no control over how and when each segment commences to turn. Further, there is a large sweep when moving from FIGS. 8a to 8i, for instance, which may not be acceptable when using the instrument as a surgical retractor.

FIGS. 9a to i start and finish with the same configuration as that of FIGS. 8a and 8i when the wire 10 is progressively tightened. However the turning sequence is controlled. When the instrument is in the position shown in FIG. 9a, the force exerted on all segments urging them from the in line position may be equal.

The first turn occurs at the distal end with the short segments 3 between the long segments 40 and 38 moving from the configuration of FIGS. 9a to 9d. When those segments 3 but each other or before abutment occurs the segments 3 between the long segment 38 and the long segment 36 start to turn to change the configuration from that of FIGS. 9d to 9e. Again when those segments 3 may abut each other or before they abut each other the adjacent distal segments start to turn. Such progressive successive turning of adjacent distal segments continues until the configuration of FIG. 9i is arrived at.

It can be seen that the change from FIGS. 9a to 9i involves only sweeping a very small area.

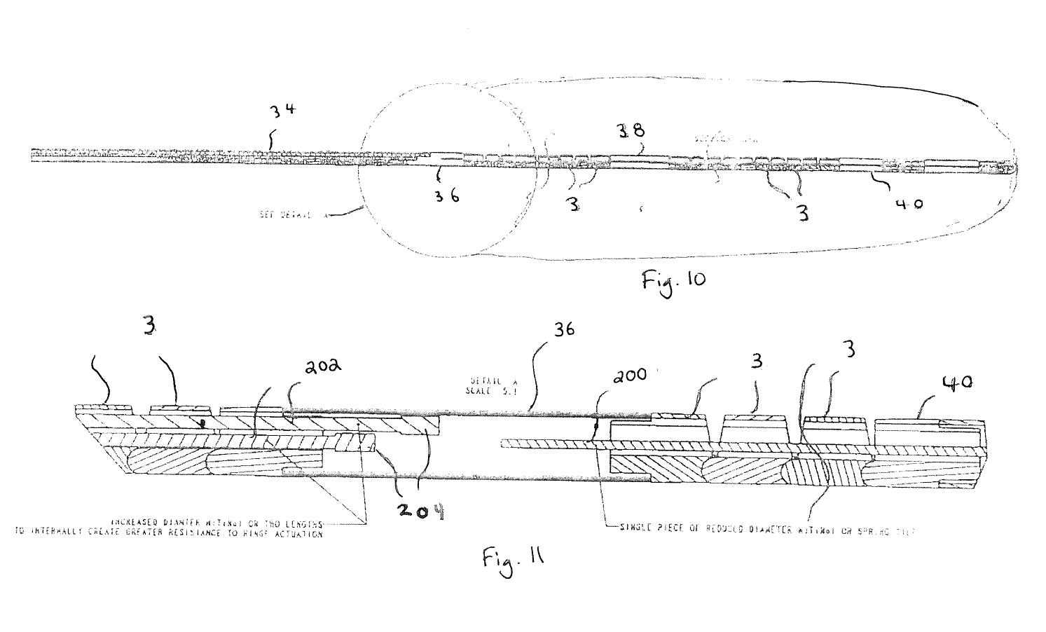

FIGS. 10 to 13 show how the control is applied. Long segments 34, 36, 38 and 40 are shown as in FIG. 7. However there may be a different number of short segments.

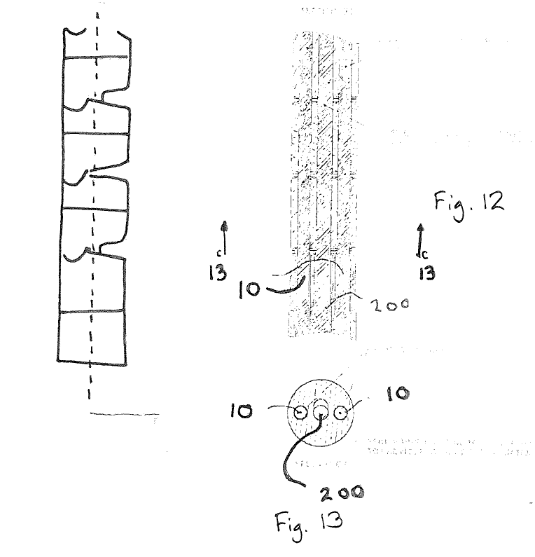

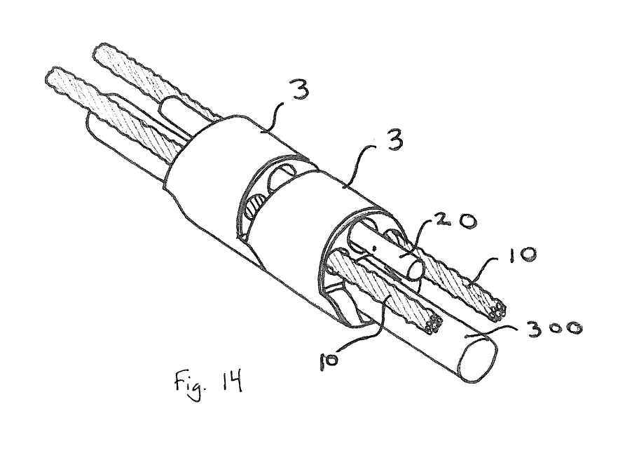

In FIGS. 11 and 12 the wire 10 that is pulled to turn the segments is not shown.

A control member 200 is connected, through the segments 3 and the long segments 38 and 40, to the distal end 40. Further control members 202 extend from the long segment 36, through the segments 3, to the long segment 34.

When the wire 10 is tightened to cause turning of the segments the control members 200 and 202 must also turn thus affording resistance to the turning (and also in due course assisting in the return of the segments to the configuration shown). The member 200 may be of the same material and/or may be of a slightly smaller diameter than each of the members 202. Accordingly the members 202 afford greater resistance to turning and the distal segments between the long segments 36 and 40 will start to turn first.

Either when all of the segments with the member 200 extending therethrough abut each other or shortly before that, the segments 3 with the members 202 extending through them will start to turn, overcoming the resistance afforded by the members 202.

Only one end of the members 202 is shown. They are provided at each end with enlarged heads 204. This stops the members 202 from moving out of the segments that they control. It also allows for the members to be straight and curved without the heads abutting the segments to restrict turning as the distance between the heads at each end is greater than the distance that they occupy when the segments are at their limit of turning. Abutment of the segments may limit the extent of a turn. Alternatively or additionally abutment of the heads 204 at each end of one or both members 202 with segments at the end of a portion that is being controlled may limit the extent of a turn.

The control members comprise spring steel or a memory metal such as NiTiNoL which may comprise Ni:Ti 50:50 Nickel Titanium alloy. Whilst the members 200 and 202 are shown as being of the same or similar diameter and material they may be of different material. Alternatively they may be of different cross sectional dimensions such as of different diameters. Alternatively the members 202 may be connected along their length.

FIGS. 12 and 13 show the location of the wire 10 and the control member 200.

Referring back now to FIGS. 9a to g, the resistance of the control member or members 200, 202, 204 and 206 progressively increases. However, if desired, the control members could be arranged in an order such that 200 affords the greatest resistance, then 204, then 202 and then 206. In this manner the sequence of movement of any bendable member can be controlled.

In an alternative, the control member 200 affording low resistance could be located through segments at the distal region, causing that region to turn first, with a stiffer control member 202 extending through segments at a proximal region.

Similarly in FIGS. 5 and 6, the control member 200 may afford less resistance than the control member 202.

With the retractors that have been described, each require a cut to be made and a sealable port to be inserted into that cut. This is time consuming and can leave an unsightly scar. Furthermore, the instrument has to be threaded through a tube that itself extends through the port. That is because the instruments are designed to flex and whilst they might be able to be locked in a retractor position they are not able to be locked in a straight position. Consequently if they were not inserted through a guide tube they would tend to bend in an undesirable and unpredictable way.

FIG. 14 is a perspective view of part of an endoscopic instrument according to the present invention. It will be appreciated that the segments 3 could be according to any of the previously described figures.

As previously described control members 10 can cause the segments 3 to turn and can assist in the return of the segments to the straight configuration. A control member 200 extends through the segments, again to operate the segments as previously described. The control member may be of memory metal.

In FIG. 14 though there is a rigid rod 300 that extends through the segments. The rod extends to the distal end or the distal end region of the instrument. The rod 300 may extend through at least some and preferably all of the segments. The rod 300 is able to slide through the segments and may be able to be caused to slide through the segments when the instrument is located in the stomach. For instance as shown in FIG. 1 the rod 300 may extend beyond the handle 6 and may include a grip 302.

In use an operative will have the rod inserted through the segments. Then, rather than making a cut and using a port and tube, the instrument can be forced through the body cavity by a sharp end 304 shown in FIG. 1 piercing through the stomach wall.

The rod 300 prevents the segments from moving relative to each other. This is particularly important when the segments are of a small diameter such as 3 mm. Furthermore it allows the segments to be lightweight and of a small diameter as the rigidity in the straight configuration is provided by the rod.

Once in position the operative can withdraw the rod either completely or partially by grasping the grip 302 and pulling.

If desired, in order to assist in the withdrawal of the retractor the rod can be pushed back through the segments to assist in their alignment or to ensure alignment in the straight direction.

The rod may be of steel such as stainless steel or titanium or any other rigid material.

Whilst the rod is shown as being cylindrical in cross section the rod may have any directed cross-section.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

What is claimed is:

1. An endoscopic instrument comprising an elongate portion having a first part moveable from a first configuration to a second, different configuration, the instrument comprising a stiffener extending across a region that the first part is moveable from the first to the second configuration, the stiffener being moveable from a first position in which movement between the first and second configurations is inhibited and a second position in which movement between the first and second configurations is permitted.

2. An instrument as claimed in claim 1 in which, in the second position, the stiffener is moved in an elongate direction of the instrument such that the stiffener does not extend across the first and second parts.

3. An instrument as claimed in claim 2 in which, in the second position, the stiffener is withdrawn from the elongate portion.

4. An instrument as claimed in claim 1 in which the stiffener extends to a distal region of the instrument when in the first position.

5. An instrument as claimed in claim 1 in which the stiffener is slidably mounted within the elongate portion.

6. An instrument as claimed in claim 1 in which the stiffener is of round cross-section along the majority of its extent.

7. An instrument as claimed in claim 1 in which the stiffener is a non-circular cross-section along the majority of its extent.

8. An instrument as claimed in claim 1 comprising a plurality of parts at different elongate extents along the instrument each moveable from a first configuration to a second different configuration in which the stiffener, in the first configuration, extends across all of the plurality of parts.

9. An instrument as claimed in claim 1 comprising an actuator arranged to urge the plurality of or each part from the first to the second positions.

10. An instrument as claimed in claim 9 in which the actuator comprises at least one wire extending along the elongate portion which wire is arranged to be tightened to effect movement from the first to the second configuration.

11. An instrument as claimed in claim 1 in which the elongate portion has a second part at a different elongate extent along the elongate portion than the first part which second part is also moveable from a first configuration to a second, different configuration, the stiffener being movable from a first position in which it extends across the regions that the first and second parts are movable from the first to the second configuration to the second position in which the stiffener does not extend across the first and second parts, at least one of the parts including a control whereby a force required to move the first part from the first configuration towards the second configuration is less than the force required to move the second part from the first configuration towards the second configuration.

12. An instrument as claimed in claim 1 comprising a piercer at a distal end.

13. An endoscopic instrument comprising an elongate portion having a first part moveable from a first configuration to a second, different configuration, the instrument comprising a stiffener extending across the region that the first part is moveable from the first to the second configuration, the stiffener being moveable from a first position in which movement between the first and second configurations is inhibited and a second position in which the stiffener is moved in an elongate direction of the instrument such that the stiffener does not extend across the first and second parts and in which movement between the first and second configuration is permitted, the stiffener being slidably mounted within the elongate portion, the instrument comprising a piercer at a distal end.

14. A method of using an endoscopic instrument comprising piercing the wall of a body with a piercer at the distal end of the instrument and inserting an elongate extent of the instrument through the wall and moving a stiffener that is slidably mounted within the elongate portion and that extends across a first part that is moveable from a first configuration to a second different configuration from a first position in which the movement of the first part is inhibited to a second position in which the stiffener does not extend across the first part and in which the movement of the first part is permitted.

15. A method as claimed in claim 14 comprising, when the stiffener is in the second position, tightening at least one wire that extends along the elongate portion to effect the movement from the first to the second configuration.

16. A method as claimed in claim 14 in which the elongate portion includes a second part at a different elongate extent along the elongate portion than the first part which is also movable from a first configuration to a second, different configuration comprising withdrawing the stiffener from both the first and second parts, at least one of the parts including a control and exerting a force on the first and second parts such that the force required to move the first part from the first to the second configuration is less than the force required to move the second part from the first configuration towards the second configuration.

17. A method as claimed in claim 14 using an instrument as claimed in claim 1.
